(19) United States
(12) Patent Application Publication
(10) Pub. No.: US 2018/0368749 A1
(43) Pub. Date: Dec. 27, 2018

(54) IMPROVEMENTS IN AND RELATING TO MUSCLE STRENGTH MEASURING DEVICES

(71) Applicant: PRECISION BIO-MECHANICS LIMITED, Hamilton (NZ)

(72) Inventor: Siamac RASTKAR, Hamilton (NZ)

(21) Appl. No.: 16/061,296

(22) PCT Filed: Dec. 12, 2016
(86) PCT No.: PCT/NZ2016/050194

§ 371 Date: Jun. 11, 2018

(30) Foreign Application Priority Data
Dec. 11, 2015 (NZ) 715070

(51) Int. Cl. A61B 5/22; A61B 5/00

(52) U.S. Cl. CPC A61B 5/224; A61B 5/4519; A61B 5/1107; A61B 5/6828; A61B 5/6829

(57) ABSTRACT

A muscle strength measuring device which includes at least one limb contact portion (LCP); at least one load cell connected to the LCP(s) so as to detect an applied load to the LCP(s); wherein, in use, a person pushes or pulls the LCP with one or more limbs, and the load cell measures the force applied to the LCP; and wherein the device includes at least one resistance element and the LCP is attached to the resistance element and is configured to move along a pre-defined linear path within the device.

### TECHNICAL FIELD

This invention relates to improvements in and relating to muscle strength measuring devices.

### BACKGROUND ART

There is a need in the art for a portable lightweight device which is capable of measuring and recording the strength of a muscle or muscle group in uniform manner for an individual—so muscle strength can be accurately gauged, at different points in time or over different periods of time.

The uniformity of measurement, when assessing muscle strength for an individual, is important so as to ensure there is no, or at least minimal, variability in recorded muscle strength occurring merely because of variables in how the machine operates.

In the fields of medicine and alternative medicine, the effects of a treatment can, to a certain extent, be subjective. This often means effective treatments are overlooked, or can be unjustly maligned, without any proper justification—and fall into non-use, or be simply labelled a fringe treatment option, and not given much weight by the medical profession.

One objective way to measure the efficacy of a treatment which involves the musculoskeletal system of a human/or animal is measuring the strength of a muscle or muscle group:
immediately prior to a treatment being applied to a human subject; and then
immediately post a treatment being applied to said human subject.

Whilst the human subject may be referred to as a patient, in this specification, the use of the term patient should not necessarily be seen as limiting, as athletes or other groups of people, may want to use the present invention to gauge performance or other physical attributes over time.

It would also be useful if there could be provided a way to explore other new methods of treatment to see if they are more beneficial than the conventionally accepted form of medical treatment or alternative form of medicine.

It is an object of the present invention to address the foregoing problems or at least to provide the public with a useful choice.

All references, including any patents or patent applications cited in this specification are hereby incorporated by reference. No admission is made that any reference constitutes prior art. The discussion of the references states what their authors assert, and the applicants reserve the right to challenge the accuracy and pertinency of the cited documents. It will be clearly understood that, although a number of prior art publications are referred to herein, this reference does not constitute an admission that any of these documents form part of the common general knowledge in the art, in New Zealand or in any other country.

Throughout this specification, the word “comprise”, or variations thereof such as “comprises” or “comprising”, will be understood to imply the inclusion of a stated element, integer or step, or group of elements integers or steps, but not the exclusion of any other element, integer or step, or group of elements, integers or steps.

Further aspects and advantages of the present invention will become apparent from the ensuing description which is given by way of example only.

### DEFINITIONS

The term ‘microprocessor’ as used herein refers to the microprocessor itself together with any associated peripheral devices The term microprocessor' will thus be seen to include a smartphone. Peripheral devices may include but should not be limited to:
memory;
display screen;
user interface;
power source;
bluetooth or wifi component(s).

The term ‘immediately’ as used herein refers to there being an action taken straight after a previous action or event. For further clarity of the term ‘immediately’ as used herein it should be taken to refer to an action being taken within 2 minutes and no longer than 5 minutes.

### SUMMARY OF THE INVENTION

According to one aspect of the present invention there is provided a muscle strength measuring device which includes:
at least one limb contact portion (LCP);
at least one load cell connected to the LCP(s) so as to detect an applied load to the LCP(s);
wherein, in use, a person pushes or pulls the LCP with one or more limbs, and the load cell measures the force applied to said LCP;
wherein the device includes at least one resistance element and the LCP is attached to said resistance element and is configured to move along a pre-defined linear path within the device.

According to another aspect of the present invention there is provided a muscle strength measuring device substantially as described above wherein at the at least one load cell measures the maximum force applied to one LCP.

According to the third aspect of the present invention there is provided a muscle strength measuring device substantially as described above wherein at the at least one load cell measures the maximum force applied to two LCPs.

According to the fourth aspect of the present invention there is provided a muscle strength measuring device substantially as described above wherein the device includes at least one resistance element and the LCP is attached to said resistance element and is configured to move along a pre-defined linear path within the device.

According to the fifth aspect of the present invention there is provided a muscle strength measuring device substantially as described above wherein the resistance element progressively applies more resistance to movement of the LCP as the LCP moves away from a start position; and wherein the load cell measures the maximum resistance encountered by the LCP each time it travels along said linear path.

According to the sixth aspect of the present invention there is provided a muscle strength measuring device substantially as described above wherein, the load cell is connected to a microprocessor wherein the microprocessor receives the load cell data and records the measured load for a given subject.

According to the seventh aspect of the present invention there is provided a muscle strength measuring device substantially as described above wherein the load cell transfers data to a microprocessor for:
the pre-treatment measured load for a subject; as well as,
the post treatment measured load for said subject.

According to the eighth aspect of the present invention there is provided the use of a device to measure and record a patient or person's strength before and after a treatment.

Preferably, the device is used immediately (i.e. just) before a treatment or series of treatments and immediately (i.e. just) after a treatment or series of treatments.

According to an ninth aspect of the present invention there is provided a use of a muscle strength measuring device substantially as described above to measure and record the, range of force applied to an LCP by a user during a specified period of time.

### BRIEF DESCRIPTION OF THE DRAWINGS

Further aspects of the present invention will become apparent from the ensuing description which is given by way of example only and with reference to the accompanying drawings in which:

FIG. 1 is a perspective view of the muscle strength measuring device of the present invention according to one preferred embodiment thereof;

FIG. 2 is a side view of the muscle strength measuring device of FIG. 1;

FIG. 3 is a partial cutaway underside view of LHS of the muscle strength measuring device of FIG. 1 wherein the lower housing has been removed, and the left LCP is in the left most position;

FIG. 4 is a partial cutaway underside view of the muscle strength measuring device of FIG. 1 wherein the upper housing has been removed, and the left LCP is in the left most position; and

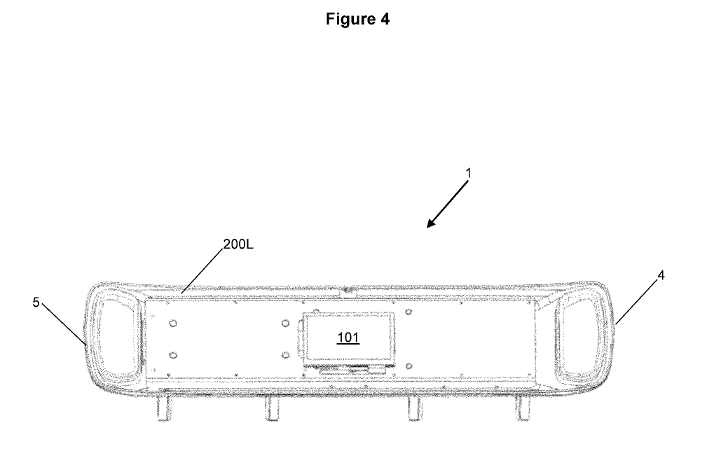

FIG. 5 is a partial cutaway view as in FIG. 3 but of the RHS wherein the right LCP is in the rest state prior to being moved by a patient.

### BEST MODES FOR CARRYING OUT THE INVENTION

With respect to the Figures there is provided a muscle strength measuring device 1 which has a body portion 100, a right hand side LCP 2, and a left hand side LCP 3.

The body portion 100 has a plastic housing 200 with an aperture 1000 for a display screen 101 on the top surface thereof along with hand holds 4, 5 for carrying or holding the device 1. Situated behind the screen 101 is a microprocessor (not shown) and associated printed circuit board (PCB) (also not shown). The plastic housing is made up of upper and lower halves 200U and 200L.

The LCP 2 and LCP 3 are both in the form of U-shape members with two downwardly projecting arms 2a, 2b and 3a, 3b linked by connector arms 2c, 3c.

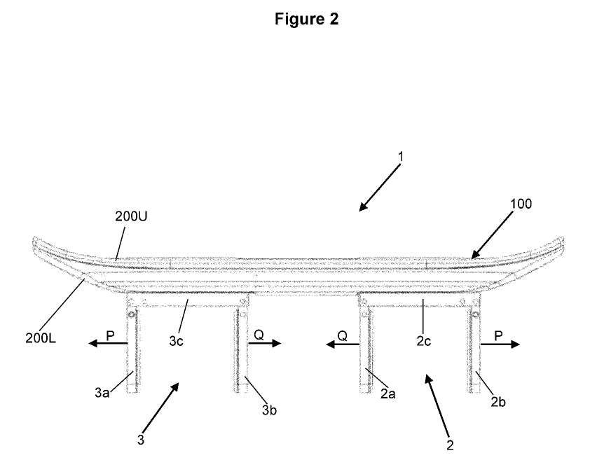

The connector arms 2c,3c of U-shaped LCP 2 and LCP 3 are each configured to slidingly engage with a linear track 50 located on the chassis 500 to be moved in directions Q and P as shown in FIG. 2.

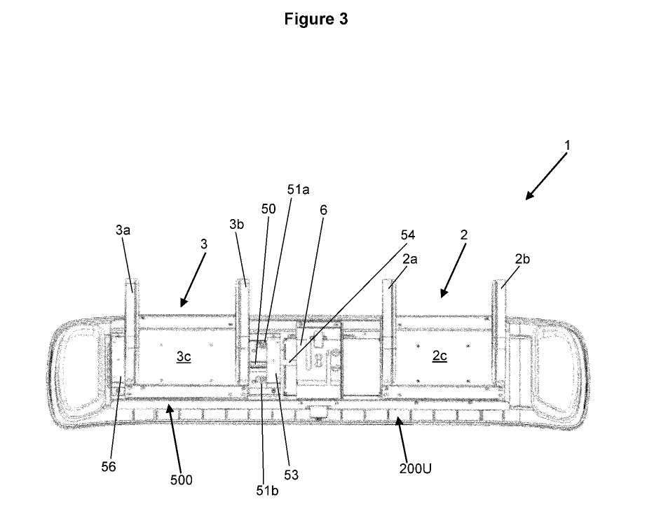

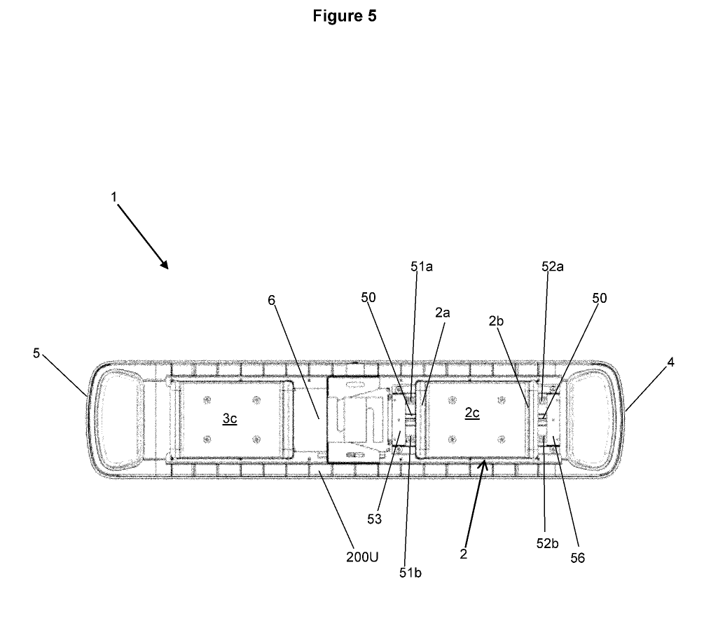

The LCPs 2 and 3 are each connected to a pair of proximal resistive elements in the form of coil springs 51a,b on one side thereof and a pair of distal resistive elements in the form of coil springs 52a,b (not shown) on the other side thereof—as can be seen in FIG. 3 and FIG. 5. A support block 53 is also connected to the other end of coil springs 51a,b and a further support block 56—located on the left and right sides of the LCP's 2 and 3 respectively—is connected to the other end of coil springs 52a,b.

FIG. 3 shows the LCP 3 when it has been pushed along track 50 to its leftmost position.

FIG. 5 by way of contrast shows the LCP 2 in its rest position.

The muscle strength measuring device 1 also has a load cell 6 which is connected to support block 53 via pressure sense bolt 54 to measure the load applied to LCP 3. Although, not shown in the embodiment depicted in FIG. 5 (it will be appreciated that a further load cell and/or pressure sense bolt could also be employed to measure the load applied to LCP 2. It will be appreciated that if the load applied to LCP 2 can also be measured along with the load on LCP 3: this enables individual LCP or collective LCP applied loads to be measured. In use, a person either attempts to:
squeeze LCP 2 and LCP 3 towards one another along the linear track (see arrows in FIG. 2); or
spread LCP 2 and LCP 3 away from one another along the linear track (see arrows in FIG. 2);.

It will be appreciated that in some embodiments only one LCP may be connected to a load cell.

### Example 1

In this example a patient goes to a chiropractor with lower back pain.

The chiropractor fits the muscle strength measuring device to the patient's ankles when they are lying in a prone position and asks the patient to:
First, push (spread) their legs outwards with all their strength; and
Second, pull (squeeze) their legs inwards with all their strength.

The device measures and records the pre-treatment force applied to the LCPs by the patient when pushing and pulling.

The chiropractor then treats the patient's lumbar joints, and then, fits the device to the patient.

Immediately following the treatment (i.e. just after). The patient then repeats the above to exercises to measure and record the post-treatment force applied to the LCPs.

In this example the patient recorded:

### Pre-Treatment

A pre-treatment spread (abduction) of 5 Kg
A pre-treatment squeeze (adduction) of 3 Kg

### Post-Treatment

A post-treatment spread (abduction) of 8 Kg
A post-treatment squeeze (adduction) of 7 Kg

In addition to any subjective feedback from the patient, the recorded measurements objectively show that, the treatment was useful in treating the lower back pain.

### Example 2

### Measuring Treatment Efficacy

In this example a patient goes to a chiropractor with neck pain.

The chiropractor determines the patient has subluxation of the neck and needs an adjustment.

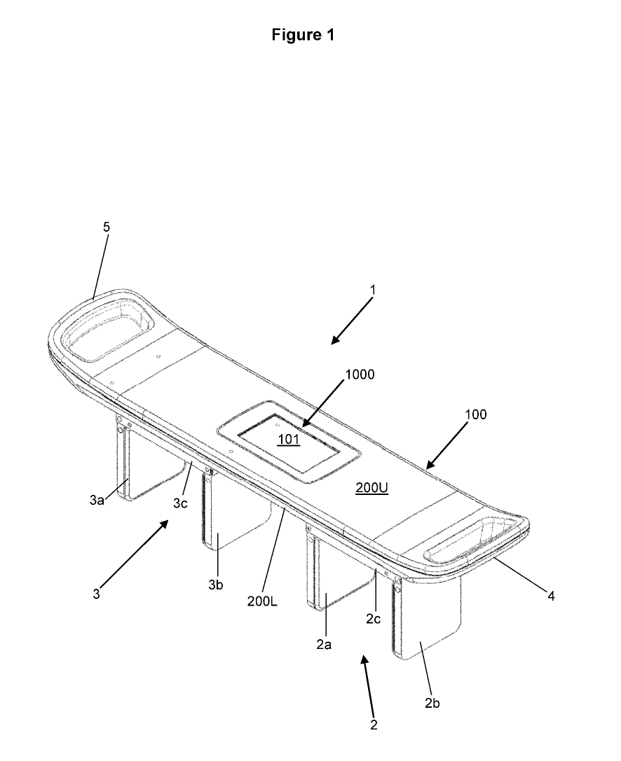

The chiropractor then fits the device shown in FIG. 1 to the ankles of a patient in a prone position and requests they:
first push (spread) their legs outwards with all their strength; and
second pull (squeeze) their legs inwardly with all their strength.

The device measures and records the pre-treatment force applied to the LCPs by the patient when:
spreading the LCPs; and when
squeezing the LCPs.

Immediately following (i.e. just after) the pre-treatment measurements have been recorded the chiropractor then treats the patient's neck.

Then immediately following (i.e. just after) the treatment the chiropractor fits the device to the patients ankles and repeats the spread and contraction exercises outlined above—to measure and record the post treatment force applied to the LCPs by the patient.

In this example the patient recorded:

### Pre-Treatment

a pre-treatment spread (abduction) 10 kg
a pre-treatment squeeze (abduction) 9 kg

### Post-Treatment

a post-treatment spread (abduction) 20 kg
a post-treatment squeeze (abduction) 17 kg

As can be seen, the treatment has seen an improvement in the post-treatment strength over the pre-treatment strength objectively indicating that the treatment has been effective.

The microprocessor associated with the muscle strength measuring device records the measured loads and wirelessly transmits this data to the patient's file on the chiropractor's remote microprocessor system.

### ALTERNATE WAYS FOR IMPLEMENTING THE INVENTION

The muscle strength measuring device may include a body portion which contains the various components comprising the device.

Generally the body portion may include an internal chassis and an external housing. However, this should not be seen as limiting.

The LCP may come in a variety of different forms provided it can be quickly and easily placed on a limb to enable operation of the muscle strength measuring device.

In one preferred embodiment the LCP may be in the form of a substantially U-shaped member which can surround a portion of a person's limb.

In another preferred embodiment the LCP may be in the form of a substantially C-shaped member.

In a still further preferred embodiment of the present invention the LCP may be in the form of a pair of opposed arms with the gap there between being spaced to receive a portion of a person's limb.

In one further embodiment the LCP may be in the form of a strap assembly including a strap and quick release cam buckle.

In one preferred embodiment the LCP may be configured to fit about a person's ankle or wrist.

In some embodiments the LCP may be fixed in place with respect to the muscle strength measuring device. Furthermore the LCP may be constructed to flex or pivot under an applied load.

In one preferred embodiment at least one LCP may be configured to form, or may be attached to a carriage, which engages and moves along a linear track, located on the muscle strength measuring device.

In another preferred embodiment at least one LCP may have a proximal portion thereof configured to fit within, or locate on, at least one track or channel, forming part of the muscle strength measuring device. In some embodiments there may be one or more pair of wheels which run along the track or channel.

The load cell may be any load cell which can measure force and/or can relay this data to a microprocessor and/or display apparatus.

In one preferred embodiment the load cell may be directly connected to the microprocessor which relays the measured load to a display apparatus also located on the muscle strength measuring device.

In another preferred embodiment the load cell may have its own associated display registering the measured load.

In another embodiment the load cell may wirelessly transmit the data to a remote microprocessor and/or display apparatus.

The resistance element may be any material or contrivance which is capable of being moved in a direction away from start point but resists such movement.

In one preferred embodiment the resistance element may be in the form of an elastic member which needs to be stretched in order for the LCP to move along the linear path.

In another preferred embodiment the resistance element may be in the form of a coil spring which needs to be extended in order for the LCP to move along the linear path.

In a further preferred embodiment the resistance element may be in the form of a compression spring.

In some preferred embodiments there may be at least one resistance element located on either side of the LCP.

It will be appreciated that a resistance element and a movable LCP are not requirements of the present invention as applied load can be measured from a stationary arm of and LCP against which a person is exerting a muscular force. The movement and resistance elements coupled with the LCP are more to give a user a perception they are doing work.

The microprocessor may come in a variety of different forms without departing from the scope of the present invention.

In some preferred embodiments the microprocessor may be within the device and directly coupled to the load cell.

In some preferred embodiments the microprocessor may be remote from the load cell and the device.

In one preferred embodiment the microprocessor is remote from the muscle strength measuring device and wirelessly receives data from the load cell and stores said data in a database.

The specified period of time during which the load applied to an LCP may be measured for a person may vary depending on what information is required and for what purpose.

In one preferred embodiment the specified period of time may be 5-10 seconds of applied pressure to an LCP over which the force recorded each second is measured. However, specified fine periods for applying force and measuring force are envisaged.

It is envisaged the range of strength recorded for a patient during a period of time, may come in useful for identifying the levels of person's neurological state, over a short period of time after a treatment.

The invention may also be said broadly to consist in the parts, elements and features referred to or indicated in the specification of the application, individually or collectively, in any or all combinations of two or more of said parts, elements or features.

Aspects of the present invention have been described by way of example only and it should be appreciated that modifications and additions may be made thereto without departing from the scope thereof as defined in the appended claims.

1-7. (canceled)

8. A muscle strength measuring device which includes:
at least two limb contact portions (LCPs);
at least one load cell connected to the LCPs so as to detect an applied load to the LCPs;
wherein the device itself also includes at least one resistance element and the LCP are attached to said resistance element and so as to move inwardly or outwardly along a pre-defined linear path within the device;
wherein, in use, a person squeezes or spreads the LCPs with two limbs, in diverging and converging directions and the load cell measures the force applied to said LCP, to test abduction and/or adduction; and
wherein the at least one load cell measures the maximum force applied to two LCPs.

9. The muscle strength measuring device as claimed in claim 8 wherein the resistance element progressively applies more resistance to movement of an LCP as the LCP moves away from a start position; and wherein the load cell measures the maximum resistance encountered by the LCP each time it travels along said linear path.

10. The muscle strength measuring device as claimed in claim 8 wherein the load cell is connected to a microprocessor wherein the microprocessor receives the load cell data and records the measured load for a given subject.

11. The muscle strength measuring device as claimed in claim 10 wherein the load cell transfers data to a microprocessor for:
the pre-treatment measured load for a subject; as well as,
the post treatment measured load for said subject.

12. The use of a muscle strength measuring device as claimed in claim 11 to measure and record the, range of force applied to an LCP by a user during a specified period of time.
